(19) United States
(12) Patent Application Publication
(10) Pub. No.: US 2021/0356460 A1
(43) Pub. Date: Nov. 18, 2021

(54) Method for Analyzing Immunoglobulins and Other Analytes in an Immunoassay

(71) Applicant: Charted Scientific Inc., Austin, TX (US)

(72) Inventors: Sonela Cavicke, Del Mar, CA (US); Edward Calvin, Austin, TX (US)

(73) Assignee: Charted Scientific Inc., Austin, TX (US)

(21) Appl. No.: 17/372,215

(22) Filed: Jul. 9, 2021

Related U.S. Application Data
(60) Provisional application No. 63/024,859, filed on May 14, 2020.

(51) Int. Cl. G01N 33/543; G01N 33/68; G01N 33/58

(52) U.S. Cl. CPC G01N 33/54313; G01N 21/6428; G01N 33/582; G01N 33/6854

(57) ABSTRACT

A method of assaying biological content of a sample, termed “microsphere-based binding assay”, using microspheres made of polystyrene or other materials to capture and detect in a sample one or more biomarkers that may be present in the biological sample. The method is able to measure various antibodies associated with infection or vaccine response. Microspheres can be coated with capture antigens and exposed to multiple sets of biomarkers. The biomarkers can be fluorescently active or fluorescently labeled. The method analyzes the fluorescent profile using scanning cytometry. Increased safety, reliability and efficiency is achieved over prior art methods.

### RELATED APPLICATION

This application claims the benefit of and priority to the provisional application entitled “Method for Analyzing Immunoglobulins and Other Analytes in an Immunoassay”, application Ser. No. 63/024,859 filed May 14, 2020.

### FIELD OF USE

This application contains a disclosure of a safe, reliable and accurate method for detection of antibody responses against synthetic or natural antigens. The antibodies are in samples from a human patient or animal. Detection may include detection of antibodies with the detection of additional biomarkers in the samples. Frequently, human patient or animal subjects are tested for antibody responses due to infection or vaccine. The disclosure teaches methods for safe and accurate assays without risks of cross-contamination of the numerous samples being tested. In addition, the disclosure teaches methods of sample assay which offer significantly decreased risk of infecting laboratory personnel.

### BACKGROUND OF THE INVENTION

Accurate detection of the existence of infection is critically important. Methods of detection commonly involve the collection of samples from persons and testing of the samples for the presence of pathogens or antigens to the pathogen. Also common is the detection of antibody response against current or previous infections. Efficient detection methods process multiple samples in quick succession. As these samples can be biological samples from patients, they can contain pathogens which are biohazardous. Many existing methods of analysis such as flow cytometry require aspiration of the sample using a sample probe, which creates biohazardous aerosols. After analysis, the samples are discharged into a waste container containing biohazardous waste fluids that must be disposed.

### BRIEF SUMMARY OF DISCLOSURE

A method of assaying biological content of a sample, termed here “microsphere-based binding assay”, uses microspheres made of polystyrene or other materials to capture and detect in a sample one or more target analytes that may be present in the biological sample. “Target analytes” include without limitation proteins (including antibodies), peptides, polysaccharides or nucleic acid sequences. Specifically, the method is able to measure various antibodies associated with infection or vaccine response; these antibodies are produced naturally by humans and other animals used in biological research upon exposure to a pathogen such as a virus or upon vaccination. In addition, other biomarkers which are not antibodies may also be simultaneously measured. In one such application, the microspheres are used to detect and/or quantify one or more isotypes of immunoglobulin antibodies (Ig) in biological samples specific to viral antigens, such as immunoglobulin G (IgG), immunoglobulin M (IgM), immunoglobulin E (IgE), or immunoglobulin A (IgA). In order to detect such antibodies, constituents of the pathogen or the vaccine such as fragments of a virus, e.g. viral peptides or proteins (“capture antigen”), are bound to the outer surface of the microspheres, which are then used to capture the antibodies of interest if present in the sample.

In the assay, each distinct microsphere set (differentiated via size, fluorescent properties or other distinct parameters that can be measured optically) would be coated with a distinct capture antigen, such as specific viral proteins. If present in the sample, target antibodies which bind specifically to the capture antigen then become bound to the capture antigen on the surface of the microspheres. The presence of the target antibodies in the sample may then be detected by introducing a fluorescently labeled detection antibody to the sample. In an example assay, each of two different microsphere sets (Microsphere Set 1 and Microsphere Set 2) could be coated with two different proteins from a virus, for convenience referred to as Viral Protein 1 and Viral Protein 2. Antibodies against Viral Protein 1 in the sample would bind to Viral Protein 1 on Microsphere Set 1, and antibodies against Viral Protein 2 in the sample would bind to Viral Protein 2 on Microsphere Set 2. The antibodies present in the sample might belong to any or all of the IgG isotype, the IgM isotype, or another isotype. A fluorescently labeled anti-IgG detection antibody that binds to any antibody of the IgG isotype would be added to the sample to label any IgG antibodies bound to each microsphere. During analysis, the amount of IgG antibodies against Viral Protein 1 present in the sample could be determined based on measured data, hereinafter “inferred”, by measuring the fluorescence of Microsphere Set 1. Similarly, the amount of IgG antibodies against Viral Protein 2 present in the sample could be inferred by measuring the fluorescence of Microsphere Set 2.

Because each of the different isotypes of immunoglobulins can bind to the same capture antigen, more than one isotype of antibody may be bound by each microsphere. By adding multiple fluorescently labeled detection antibodies to the sample, each of which is specific for a different isotype of antibody (e.g. IgG, IgM, IgA, etc.) and each of which is labeled with a different fluorophore, the amount of multiple isotypes of antibody against each of Viral Protein 1 and Viral Protein 2 in the sample can be independently measured provided the fluorophores used to label each of the different detection antibodies can be detected and measured independently. A well-known method for measuring multiple isotypes of antibody involves using a fluorophore for each type of detection antibody with a unique fluorescence emission profile.

It is understood that within some isotypes of immunoglobulins there are also subtypes. This method applies to the measurement of subtypes of antibodies in addition to the isotypes listed above. For brevity, this disclosure may refer to classes, isotypes, and subtypes collectively as “classes”.

An assay of this type could be used for a number of applications related to infectious diseases, including but not limited to clinical diagnostics, surveillance of the rate of infection of specific pathogens in a population of individuals, measuring the immune response of individuals to pathogens, or development of vaccines. All of these applications require a method that is safe, fast, low-cost, sensitive, and reliable.

Assays such as the types described herein have been practiced in the past using flow cytometry as the method of analyzing the microspheres. Flow cytometry provides the sensitivity required for this application but has a number of drawbacks, including:
(a) Flow cytometry requires aspiration of a liquid sample so that the sample can be analyzed, and the aspiration and processing of the sample suffers reliability problems inherent to the technology;
(b) Aspiration of the sample introduces the possibility of contamination of one sample by residues from previous samples (termed “carryover”);
(c) Aspiration of the sample can create aerosols, which are potentially hazardous to laboratory personnel in the context of analysis of samples for infectious diseases; and
(d) Flow cytometry is generally regarded as too slow and the time required to process each sample is too variable for high-throughput screening, where the volume of samples to be tested requires a high degree of automation.

This disclosure teaches a method of performing and analyzing these sample assays using laser-scanning cytometry. Laser-scanning cytometry addresses all of the listed drawbacks of flow cytometry:
(a) Samples can be analyzed in a few seconds, enabling very high throughput;
(b) Samples do not need to be aspirated and in fact can be analyzed in discrete and separate sealed vessels, eliminating carryover between samples and aerosols and release of harmful aerosols; and
(c) Optical scanning of samples in microtiter plates is generally regarded as being reliable enough and fast enough for high-throughput screening applications.

### SUMMARY OF DRAWINGS

The accompanying drawings, which are incorporated in and constitute a part of the specification, illustrate preferred embodiments of the invention. These drawings, together with the general description of the invention given above and the detailed description of the preferred embodiments given below, serve to explain the principles of the invention.

FIG. 1A illustrates prior art of capturing and quantifying a single class of antibody to a single viral protein. FIG. 1B illustrates prior art of capturing and quantifying neutralizing antibody in sample by competing with labeled neutralizing monoclonal antibody.

FIG. 2 illustrates prior art of capturing and quantifying antibodies to two viral proteins using two coated microspheres.

FIG. 3 illustrates capturing and quantifying three classes of antibodies to a viral protein using a single coated microsphere.

FIG. 4 illustrates capturing and quantifying three classes of antibodies to a viral protein using a single coated microsphere and a negative control microsphere.

FIG. 5 illustrates capturing and quantifying three classes of antibodies to a viral protein using a single coated microsphere and a positive control microsphere.

FIG. 6 illustrates capturing and quantifying two classes of antibodies to two viral proteins using two coated microspheres.

FIG. 7 illustrates capturing and quantifying three classes of antibodies to a viral protein using a single coated microsphere and another biomarker using a different microsphere.

FIGS. 8A and 8B illustrate method steps for determining concentration of analytes using microspheres in a scanning cytometer.

FIG. 9 illustrates an example list of parameters obtained for particles in a sample.

FIG. 10 illustrates classification of microspheres in a sample using measured optical parameters.

FIGS. 11A, 11B and 11C illustrate a standard curve relating particle fluorescence value to biomarker concentration in a sample.

FIG. 12 illustrates an example system for measurement of optical parameters of particles in a liquid sample.

FIG. 13 illustrates an example laser-scanning cytometer system.

FIG. 14 illustrates an enclosed sample vessel.

### DETAILED DESCRIPTION OF DISCLOSURE

A safe, rapid, reliable method to quantify multiple antibody responses in a patient due to a multivalent vaccine or exposure to pathogens having multiple epitopes, with the potential to simultaneously assay other biomarkers in the sample, would be highly desirable. The method disclosed in this disclosure includes allowing for assays to be more specific by separately detecting multiple antibody isotypes. The method enables a reduction in the potential for false negatives and false positives through the use of internal positive and negative controls which can be included on separate sets of microspheres. The disclosed method meets the speed, reliability and cost needs of high-throughput screening, thereby enabling the adoption of this technology for widespread use in combating infectious disease.

Included within this disclosure is the teaching of methods for safe and accurate assays without risks of cross-contamination of the numerous samples being tested. In addition, the disclosure teaches methods of sample assay which offer significantly decreased risk of infecting laboratory personnel.

One benefit taught by this disclosure is the utilization of scanning cytometry to enable quantification of the amount of each immunoglobulin class for each target antigen (e.g. viral protein or vaccine component) captured from a sample by each microsphere set. A unique fluorescently labeled antibody specific to each immunoglobulin class is introduced into the sample, where the fluorescent labels differ from each other either in the range of excitation wavelengths, the range of emission wavelengths, or both the excitation and emission wavelengths. For example, anti-IgG detection antibodies could be conjugated to fluorescein isothiocyanate (FITC) and anti-IgM detection antibodies could be conjugated to phycoerythrin (PE). Because the fluorescent emissions of PE and FITC have different intensities as a function of wavelength, each label can be quantified independently by measuring fluorescence at multiple wavelengths even when the two labels (PE and FITC) are attached to the same microsphere.

In order to accurately determine the concentration of each analyte within a sample using a binding assay as described above, the user may use one of several methods. A typical method involves the user first deriving a standard curve relating the average fluorescence of particles in the sample for a given set of microspheres to concentration of the analyte within the sample. This standard curve is derived by first preparing a number of standard reagents each containing the analyte of interest at a different concentration.

In an embodiment, the range of analyte concentrations in the standard reagents typically spans at least the total range of concentrations expected to be observed in an actual test sample. The binding assay as described above is performed using each of the standard reagents as a sample, and the resulting fluorescence corresponding to each analyte concentration is observed. A mathematical formula such as a polynomial may be calculated to provide a mathematical model of the relationship between fluorescence and analyte concentration with the least error over the range of expected concentration (e.g. least squares curve fitting of a fourth order polynomial). Then, the measured fluorescence for a test sample with unknown analyte concentration is compared to the mathematical model to determine the measured analyte concentration. Qualitative assays (such as determining if a sample contains or does not contain a certain analyte without attempting to measure the concentration of the analyte) can also be performed, wherein cut-offs between negative and positive responses are determined so that samples may be classified as either positive or negative by comparison to the determined cut-off values.

Additional analytes in the sample, such as secreted proteins or other biomarkers, may be simultaneously assayed using additional sets of microspheres, where each set of microspheres used is distinguishable from the other sets of microspheres based on some combination of properties that may include size, shape, opacity at certain wavelengths, and fluorescence at certain wavelengths. Each individual set of microspheres is coated with a unique capture antigen or antibody, so that each set of microspheres captures a unique type of analyte present in the sample. Multiple sets of microspheres may be simultaneously exposed to the sample. The user would determine a standard curve, as previously described, relating fluorescence on the surface of the relevant microsphere set to concentration of the target analyte for each set of microspheres added to the sample, thereby enabling simultaneous measurement of multiple analytes within a single sample.

In order to perform a binding assay and use its results to measure analyte concentration, a means of measuring the fluorescence of particles in the sample is needed. Flow cytometry and scanning cytometry are two such means.

As its name implies, flow cytometry is a technique that uses specially designed optically clear channels through which a liquid carrier transports a continuous stream of sample material to present the particles (e.g., cells or microspheres) in the sample one at a time to an optical system for measurement. The particles are typically illuminated by one or more focused lasers that illuminate only one particle at a time. The illumination may also be performed with other devices such as light emitting diodes (LEDs), arc lamps, or other light sources.

Flow cytometry is an efficient means of evaluating a large number of particles in a sample since the time to measure each individual particle is on the order of a few microseconds. It will be appreciated that the technique utilizes a continuous stream of particles. The properties that are typically recorded for each particle include forward scattered light, side scattered light, back-scattered light, and one or more colors of fluorescence used to identify the previously referenced fluorescent labels. A flow cytometer might use one, two, or more lasers to collect the desired number of measurements for each particle in the sample.

Flow cytometry suffers a number of drawbacks when used as part of a method to assay liquid samples. One drawback of flow cytometry results from measuring particles sequentially. In order to measure a large number of particles sequentially in a short period of time, the time allowed to measure each individual particle is also short. The necessity to analyze each particle quickly degrades the sensitivity of the measurement made on each particle.

A second drawback results from the method of illumination typically employed in flow cytometers. In order to provide highly uniform illumination to each particle, whose position within the optically clear channel may vary from particle to particle, a field of illumination substantially larger than the particle is used. Typically, an illumination field ten times the diameter of each particle or greater is used to illuminate each particle such that the illumination received by each particle only varies by a few percent from one particle to the next. Consequently, flow cytometers are only able to use a small percentage of the total illumination to analyze each particle. Because the illumination source is many times brighter than what is needed to illuminate a particle (between 10× and 100× brighter than the amount of light that actually illuminates the particle at any given time), the amount of stray light in the optical system is also much higher than desirable. Excess stray light interferes with the flow cytometer's ability to detect very weakly fluorescent particles, thereby degrading the sensitivity of the measurement.

A third drawback of flow cytometry is the difficulty of incorporating flow cytometers into a highly automated analysis system. Flow cytometers are prone to a number of failure modes owing to the passage of fluid through microscopic channels and the precise alignment required of the lasers used to illuminate samples. The lack of reliability precludes high levels of automation, where predictable analysis in a predictable amount of time of a large number of samples is a must.

Lastly, the method of aspirating liquid samples typically required in flow cytometry introduces cross-contamination between samples (sample carryover) and can create potentially hazardous aerosols. Attempts have been made to address these drawbacks by incorporating the liquid-handling components of the flow cytometer into disposable microfluidic devices, but these devices increase the cost of analyzing each sample as well as increase the amount of time required to process each sample. In the context of high-throughput screening, disposable microfluidic cartridges are not a viable solution.

This disclosure teaches use of scanning cytometry. Scanning cytometry, or laser-scanning cytometry, uses a microscope equipped with an optical scanning system to analyze and measure a number of cells or microspheres presented, for example, on a microscope slide for analysis. The samples are typically static; that is to say that particles being analyzed are spread out over a flat surface while being analyzed, and the optical system scans across the surface to evaluate the individual particles. The samples may be contained in discrete sample containers (such as a microtiter plate comprised of 96 individual sample wells). Each sample is separately presented and subjected to testing. In an embodiment, the slide holding the particles may be translated using a motorized stage beneath a fixed optical analysis system. Like a flow cytometer, a scanning cytometer is able to measure multiple fluorescence and light-scattering properties simultaneously.

Scanning cytometers address the illumination issues of flow cytometers by only illuminating the particle being analyzed with a focused light source (typically a laser). Through proper choice of illumination source and lenses, the illumination delivered to each particle may be as small as or smaller than the particle. These instruments can use lower power illumination sources compared to flow cytometers and have substantially less stray light than flow cytometers.

Scanning cytometers typically use an epi-fluorescent microscope as the means of delivering light to the sample and collecting light emitted by the sample. The means of illumination could be scanned across the sample by means of a movable mirror. While this configuration eliminates the problem of illuminating an excess area surrounding each particle, it still illuminates excess material in the sample above and/or below the particle of interest. To the extent that other particles or other unbound fluorescent material is present in the sample at different depths than the particle of interest, it will interfere with measurement of weak fluorescent signals from the particle and thereby reduce the sensitivity of the measurement.

Whereas flow cytometers are designed to measure particles suspended in a liquid sample, scanning cytometers are generally limited to measuring particles that have been immobilized on a flat surface. Immobilization can be achieved by placing a coverslip over the sample or by fixing the particles in the sample to a planar surface. It will be appreciated that the planar surface can be the bottom surface of a closed sample vial or container.

Disclosed herein is a method for making sensitive measurements of the quantity of one or more target analytes that are dissolved or suspended in a liquid sample. The term “analyte” used in this method means a specific chemical compound that may be present in a biological sample, including without limitation proteins, peptides, polysaccharides or nucleic acid sequences. The target analytes include at least two antibodies associated with vaccine response or an infection by a pathogen such as a virus, and may also include antibodies associated with infection by additional pathogens and/or biomarkers normally found in bodily fluids such as cytokines and other proteins or peptides.

The method described in this disclosure could be used to measure the immune response to a pathogen or a constituent component of a pathogen (such as a protein or peptide) as well as the immune response to a vaccine or a constituent component of a vaccine, where the vaccine contains proteins that are similar to or the same as proteins found in the pathogen to which the vaccine is meant to provide immunity.

It will be appreciated that the teaching of this disclosure includes creation of the ability to measure immune response in a sealed sample container while being able to measure the different isotypes of antibody for each capture antigen. This has the advantages of safety, accuracy and reliability.

The method described in this disclosure could also be used to measure the immune response to multiple viral proteins associated with the same pathogen or vaccine. The method could also be used to measure immune responses to different pathogens. The latter method would be useful in situations where multiple pathogens elicit similar immune responses from patients, and separate detection of antibodies to each pathogen provides the ability to diagnose infections with better specificity.

The method described in this disclosure could be adapted to include either or both of a positive control for an assay and a negative control. An example of a positive control would be the measurement of a protein that is expected to be present in all samples, such as total IgG in serum. By measuring the aggregate amount of all different types of IgG antibodies in a sample, the user is able to determine whether or not the correct amount of patient sample was used and thereby validate the results of the assay. In the case of a negative control, a biomarker that is not expected to be present in the sample could be assayed. If a significant amount of that biomarker is detected when analyzing the assay, then a problem with the test is indicated and the assay results would be invalidated. Alternately, a protein such as bovine serum albumin (BSA) could be coated onto the particle. In most assays, biomarkers that are typically present in the sample would not adhere to the BSA. Any fluorescence detected resulting from proteins bound to the BSA negative control would indicate a problem with the assay.

The method could be used to measure a patient response to an infection or to a vaccine that is distinct from and in addition to a direct immune response. In the case of monitoring a patient's response to a vaccine, the user could measure biomarkers that are indicative of an inflammatory response. Such a response might indicate an undesirable systemic response to the vaccine (e.g., a cytokine storm).

The scanning cytometry method taught by this disclosure could be used to prevent exposure to the user of potentially infectious pathogens, e.g., via aerosols, that might be present in a liquid sample. The method also reduces the possibility of contamination of a sample by residues from other samples or contamination by materials other than samples that might be present. Because the sample does not need to be aspirated in order to be analyzed by the method, the assay could be performed entirely within an enclosed vessel.

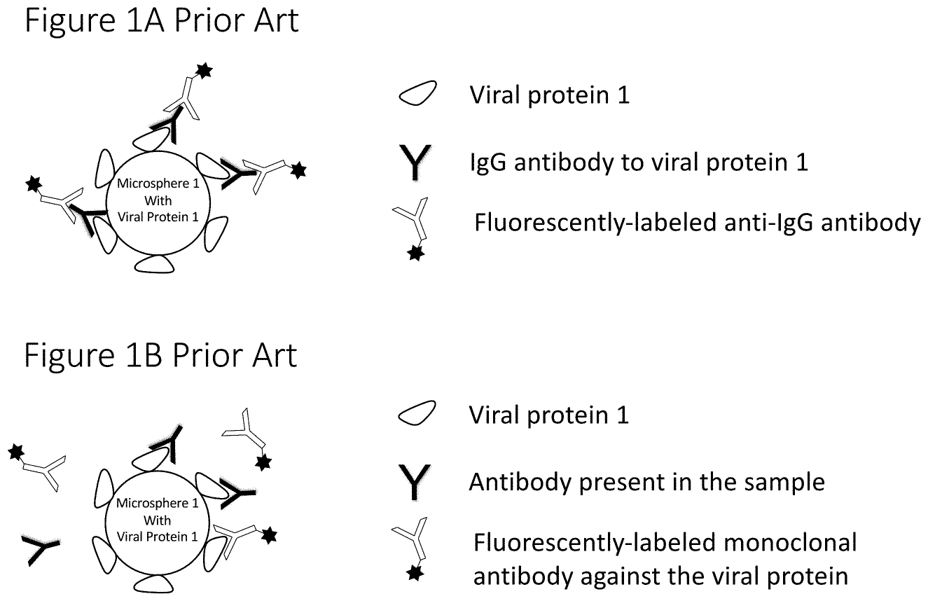

Known methods for determining the concentration of antibodies created as an immune response to a pathogen or to a vaccine is illustrated in FIGS. 1A and 1B. In FIG. 1A, microspheres are coated with proteins or peptides to which the antibodies to be measured bind. A second set of fluorescently labeled antibodies is added to the sample which binds to the target analyte. The target analyte in FIG. 1A is IgG antibody against viral protein 1. The quantity of the target analyte in the sample is inferred from the amount of fluorescence measured by illuminating the microsphere with light at a wavelength that excites the fluorescent label.

FIG. 1B illustrates a commonly used competitive assay format. As in FIG. 1A, the microspheres are coated with the protein or peptide to which the analyte to be measured binds. A fluorescently labeled version of the analyte to be measured, which is added to the sample, competes with the analyte for binding sites on the microspheres. If no analyte is present in the sample, the fluorescent signal will be high. The target analyte in FIG. 1B is antibody against viral protein 1, which could be the IgG isotype or another isotype. The fluorescently labeled version of the target analyte would be the same isotype as the target analyte. As the level of analyte in the sample increases, less of the fluorescently labeled analyte is able to bind to the microspheres resulting in a lower fluorescent signal.

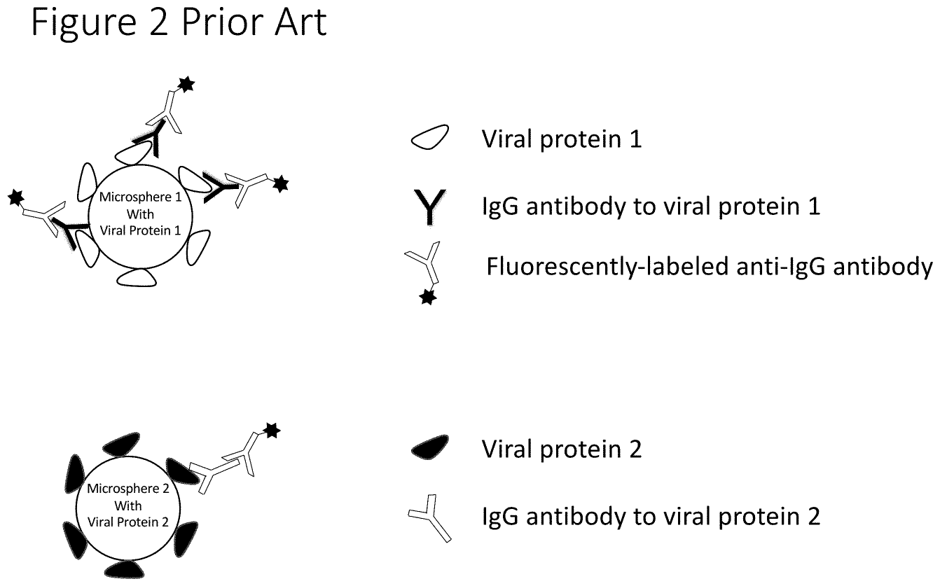

FIG. 2 illustrates a known method for determining the concentration of two different antibodies created as an immune response to two different viral proteins in a liquid sample. Different proteins are coated onto two different sets of microspheres, each set distinguishable from the other set by one or more optical parameters such as size, emission of fluorescence at a certain wavelength, or absorbance of light at a certain wavelength. The two viral proteins could be different proteins from the same pathogen or proteins from different pathogens. The viral proteins could also be proteins used as components of vaccines intended to confer immunity to the pathogen to a patient. The quantity of each antibody in the sample is determined as in the method illustrated in FIG. 1.

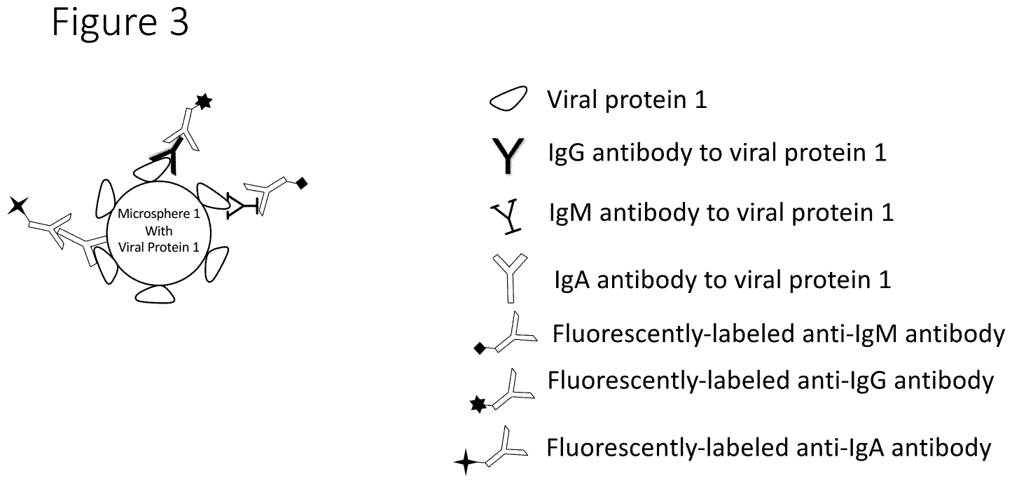

FIG. 3 illustrates a novel embodiment of the disclosed invention wherein a single set of microspheres are coated with a single viral protein. Different classes of antibodies in the sample (such as IgG, IgA, and IgM) bind to the viral protein. A fluorescent label specific to each class of antibody to be measured is added to the sample, where each label is distinguishable from the labels for other classes of antibodies based on its fluorescence excitation and emission spectrum. In FIG. 3 the fluorescently labeled antibodies are anti-IgG, anti-IgM, and anti-IgA. Each of these detection antibodies is conjugated to a different fluorophore with a different fluorescence emission profile. For example, the anti-IgG antibody could be conjugated to phycoerythrin, anti-IgM could be conjugated to fluorescein isothiocyanate, and anti-IgA could be conjugated to allophycocyanin. Each of these fluorophores has a peak fluorescent emission wavelength that is different from the other two fluorophores. The quantity of each class of antibody captured by the microsphere can be inferred from the fluorescence of the microsphere, and the concentration of that antibody in the patient sample can be inferred from the measured fluorescence. This can be achieved within a sealed sample container using a laser-scanning cytometer with improved safety, accuracy and reliability. For example, the method taught by the disclosure does not require aspiration, which can fail or cause clogging, etc.

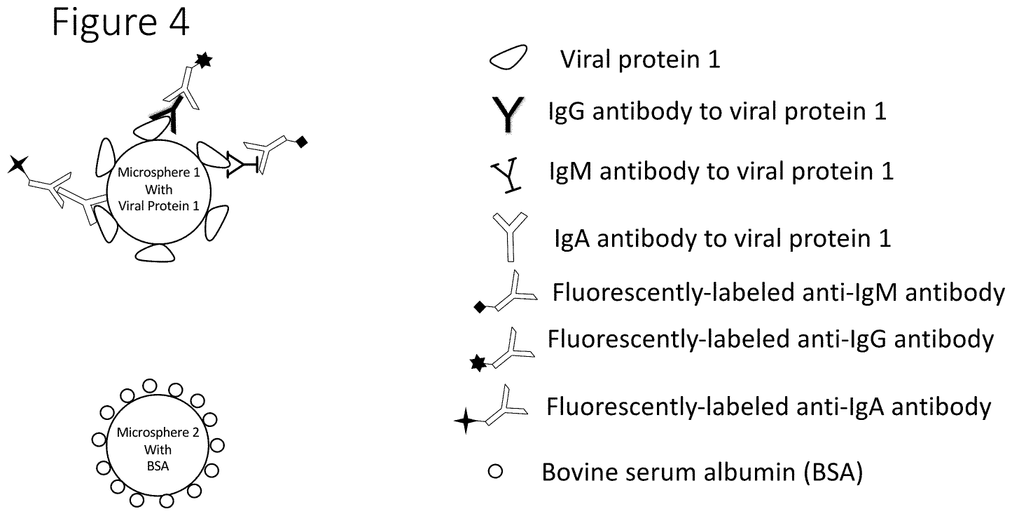

Illustrated in FIG. 4, a single set of microspheres is used to assay the concentration of different classes of antibodies to a given viral protein as in the method shown in FIG. 3. In addition, a separate set of microspheres is used as a negative control. The negative control is coated with a substance (for example BSA) which should not bind any of the target analyte in the sample and therefore the negative control microspheres should not fluoresce. If the negative control microspheres are found to be fluorescent above a predetermined maximum limit, that fluorescence indicates a problem with the assay.

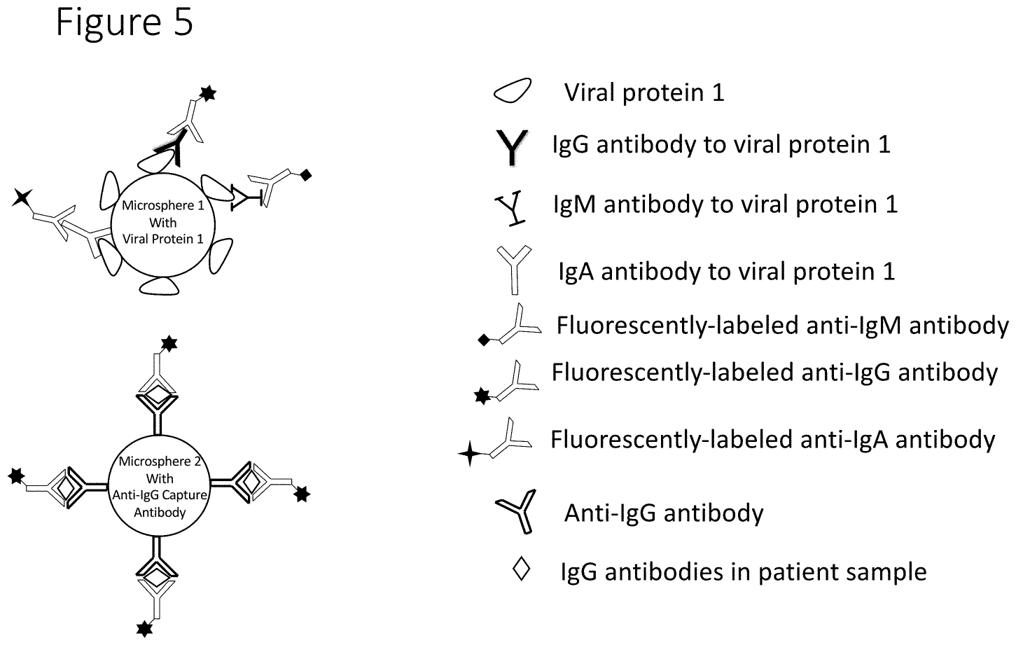

Illustrated in FIG. 5, a single set of microspheres is used to assay the concentration of different classes of antibodies to a given viral protein. In addition, a separate set of microspheres is used as a positive control. The positive control is coated with a substance (for example anti-IgG antibodies) which specifically binds a substance that should be present in all patient samples whether or not the samples contain antibodies to Viral Protein 1. Fluorescence of microspheres that are members of the Microsphere 2 set (positive control) within a predetermined range indicate that the assay has been conducted as intended and that the patient sample has been added.

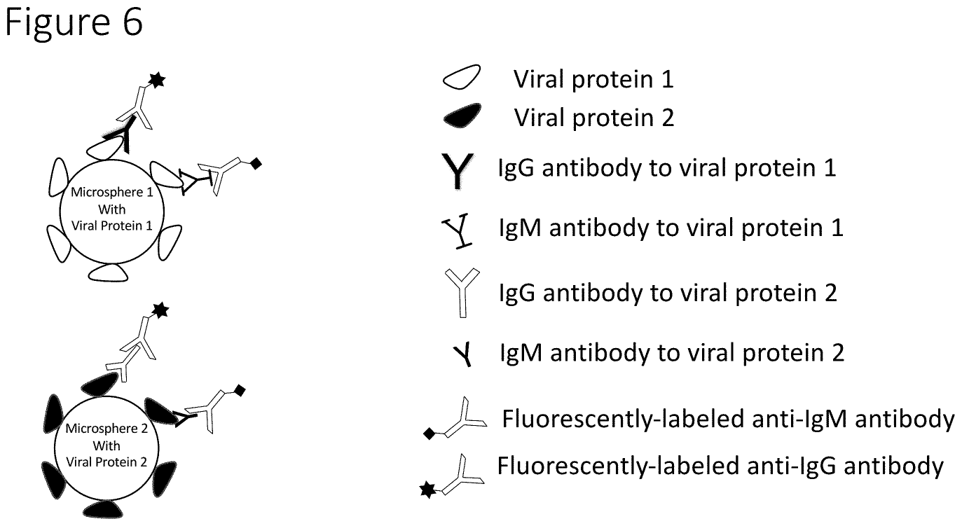

In regard to FIG. 6, a single set of microspheres is used to assay the concentration of different classes of antibodies to a given viral protein. In addition, a separate set of microspheres is added to the sample that is coated with a second viral protein. The second viral protein could be part of the same pathogen as the first viral protein or a protein pertaining to a different pathogen. The fluorescence of each of the two labels bound to the second set of microspheres is used as in the example illustrated in FIG. 3 to determine the concentration of the classes of antibodies to the second viral protein in the sample. The method illustrated in FIG. 6 enables the determination of multiple classes of antibody against multiple antigens in a single sample. It will be appreciated that simultaneous detection both of antibodies against multiple antigens while also detecting multiple classes of antibody against the multiple antigens provides useful information about an immune response.

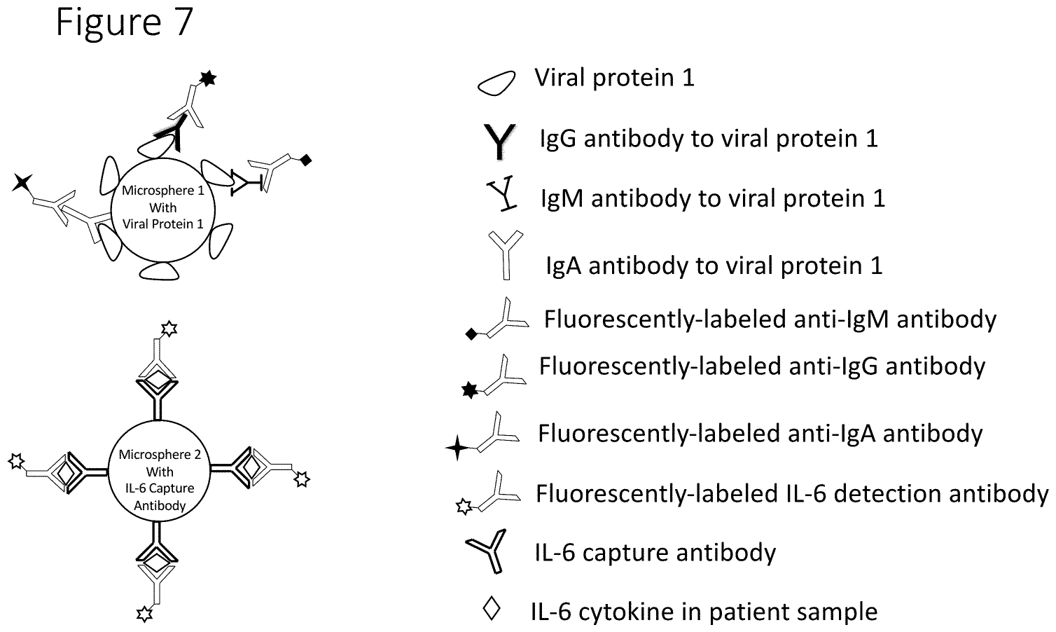

In regard to FIG. 7, a single set of microspheres is used to assay the concentration of different classes of antibodies to a given viral protein in a similar manner to the assay illustrated in FIG. 3. In addition, a separate set of microspheres is added to the sample that is coated with a capture antibody that binds specifically with a protein biomarker in the patient sample such as the cytokine Interleukin 6 (IL-6). The fluorescence of the fluorescent label bound to the second set of microspheres is used as in the example illustrated in FIG. 3 to determine the concentration of IL-6 in the sample. It will be appreciated that biomarkers other than cytokines (such as peptides or proteins other than cytokines) could also be assayed in this manner. The quantity of IL-6 in the sample could indicate a condition of the patient (such as inflammation) other than the direct immune response to the viral protein of antibody production.

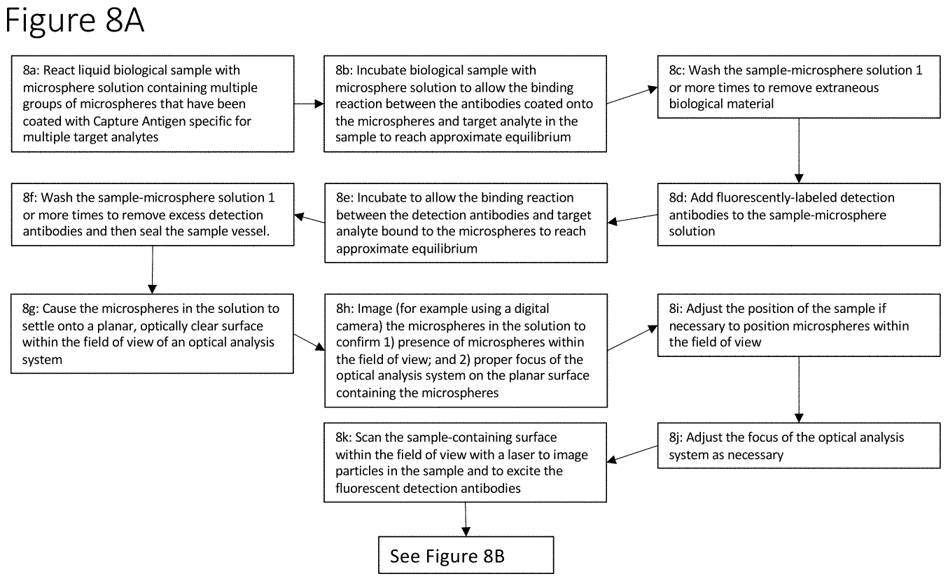

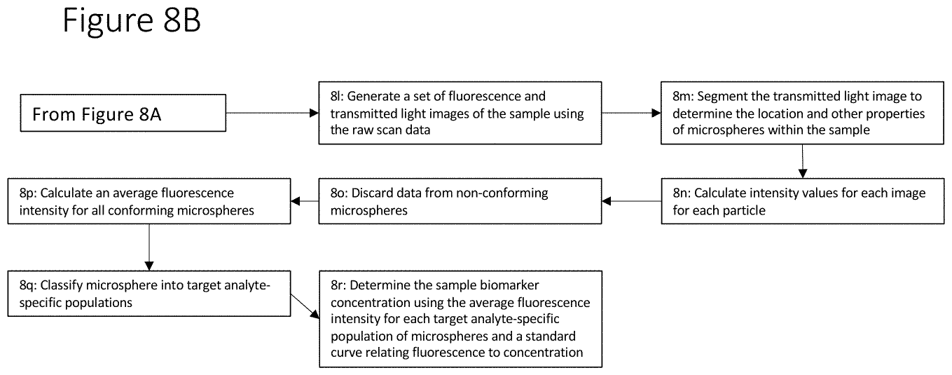

FIG. 8 describes one specific process that could be used to analyze liquid samples for analyte concentrations pursuant to the disclosure.

Because the sample is sealed within the sample vessel in step 8f, none of the subsequent steps expose personnel or equipment to biohazard, and no cross-contamination from other samples is possible during subsequent steps.

During step 8k, the sample can be scanned with one excitation wavelength (such as a laser) or multiple excitation sources at different wavelengths. This flexibility allows the generation of an arbitrarily large number of images of the sample in step 8l, where each image contains unique information about the sample's fluorescence at a particular range of wavelengths and in response to excitation at a particular wavelength or range of wavelengths. Fluorescence emission values for each combination of excitation wavelength and emission wavelength range are determined for each particle in step 8p. These values are used both to classify the particle to a particular microsphere set as well as to quantify the amount of detection antibody bound to the microsphere surface for each class of antibody. The number of fluorescence excitation wavelengths and the number of fluorescence emissions detection wavelength ranges can be selected to based on the number of fluorescence values needed for classification of the particles and for measurement of each of the antibody classes bound to the microspheres.

An alternate embodiment of the method illustrated in FIGS. 8A and 8B could omit the wash steps shown in steps 8c and 8f. The seal could be applied to the sample vessel after addition of the sample and detection antibodies, thereby containing any potential biohazards for a greater portion of the total process.

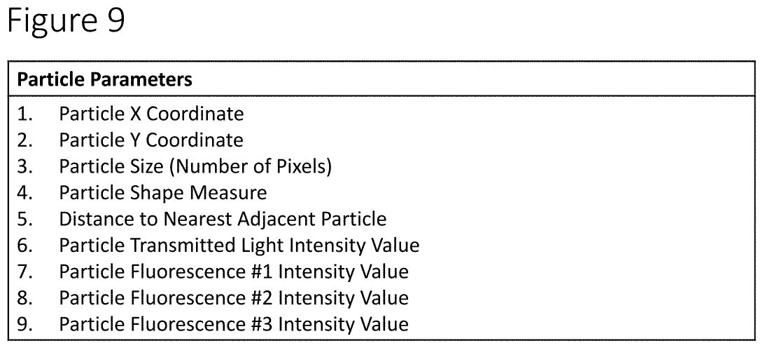

FIG. 9 lists optical parameters that could be measured for each particle in a liquid sample analyzed pursuant to the disclosure. It will be readily understood that more than three fluorescence values could be measured for each particle through the use of additional fluorescence excitation sources, additional fluorescence detection wavelength ranges, or increasing both the number of excitation sources and the number of detection wavelength ranges. It will also be appreciated that additional non-fluorescence parameters, such as the amount of light absorbed at one or more excitation wavelengths, could also be measured.

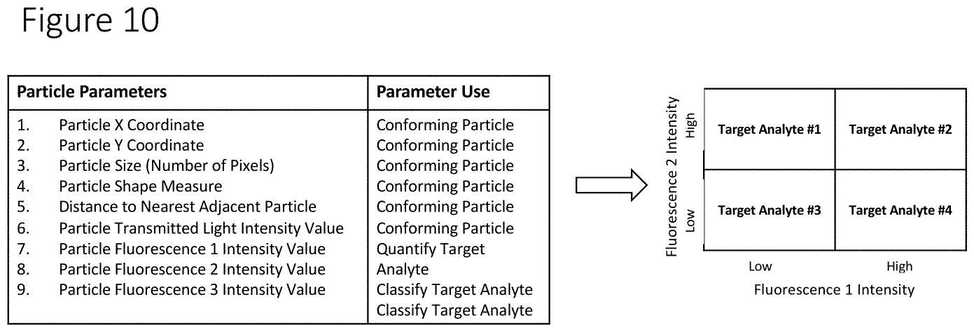

FIG. 10 illustrates the method of classifying each particle (e.g. a microsphere) in a sample. Microspheres pertaining to each target analyte are classified based on ranges of values of a subset of the parameters listed in FIG. 10. At least one fluorescence value for each particle is required to measure the amount of analyte bound to the surface of the particle. All of the other parameters may be used as classification parameters. For example, fluorescence in two different wavelength ranges could be used as classification parameters to create a two-dimensional classification space. Particles which do not classify as members of any of the sets of microspheres are deemed “non-conforming” and are not used in the calculation of any target analyte concentrations. More than two or less than two classification parameters could be used as is required for a particular assay. Creating classification rules in this manner is known art in regard to multiplexed immunoassays using microspheres.

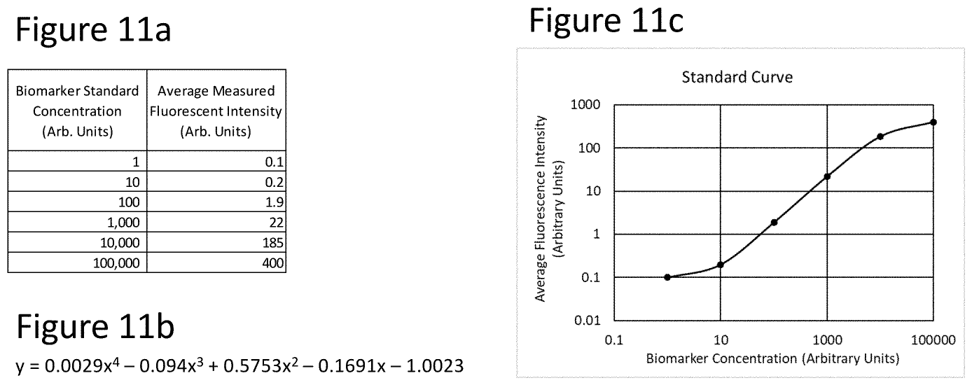

FIGS. 11A, 11B and 11C illustrate the process for estimating the concentration of a target analyte based on measured particle fluorescence values from a plurality of standard concentrations, each of which contains a known quantity of the target analyte. The fluorescence values for each of the known standards is collected, FIG. 11A, and a mathematical relationship such as the formula 11B, is derived relating measured fluorescence to analyte concentration. For samples with unknown analyte concentration, the measured fluorescence value is used to infer the analyte concentration by solving the formula to obtain the concentration. It will be appreciated that other relationships between fluorescence and concentration than the one illustrated in FIG. 11C could be derived.

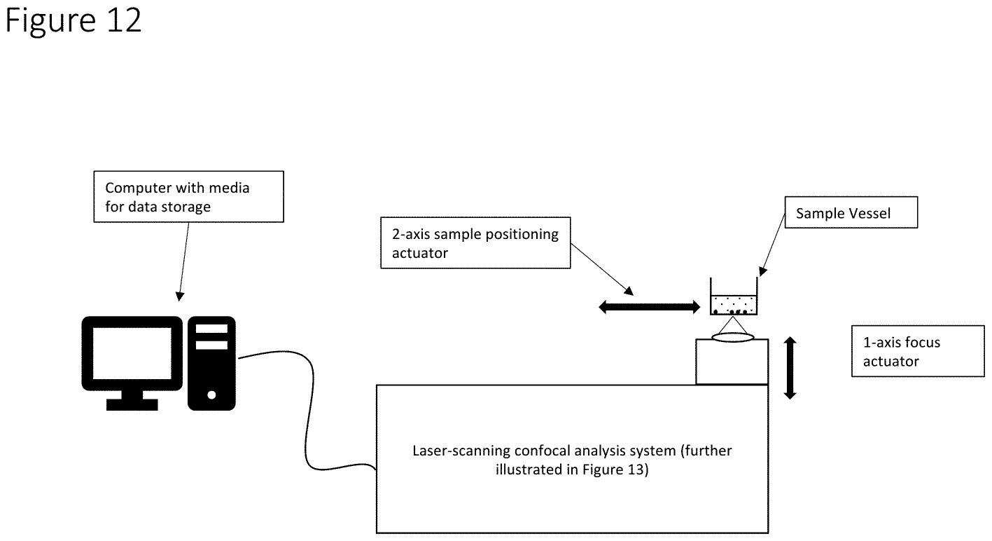

FIG. 12 illustrates one possible system that could be used to implement the methods disclosed herein. Samples are presented in a sample vessel, which has at least one planar, optically clear surface (in this example the bottom of the container). Particles in the sample settle under the force of gravity onto the planar surface. The sample vessel is positioned so that it can be scanned by the laser-scanning confocal analysis system. The laser-scanning confocal analysis system measures the optical parameters for each particle in the sample and then communicates these values to the computer. Software stored on the computer uses the measured optical parameters to classify particles in the sample and determine concentrations of analytes in the sample.

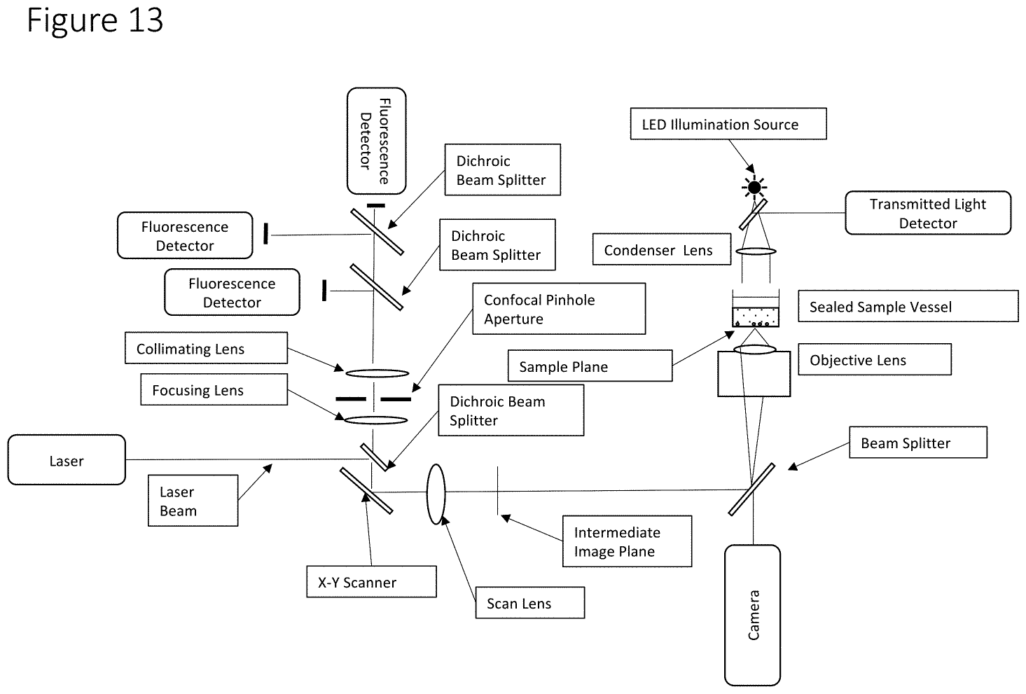

FIG. 13 illustrates a laser-scanning cytometer system that could be used for analysis of particles in a liquid sample as described in this disclosure. In this example, a single laser is used to illuminate particles in the sample. A transmitted light detector is used to measure absorption of light at the laser wavelength, and particle size and shape can be derived from the measured absorption of light at different physical locations within the sample. Three fluorescence detectors measure fluorescence at different wavelengths emanating from particles in the sample. A scanning system is used to focus the laser beam and scan it across the sample, resulting in a 2-dimensional image of the particles lying in the sample plane for each of the transmitted light detector and the three fluorescence detectors. A LED illumination source and a camera (such as a CMOS or CCD detector) are used prior to scanning the sample with the laser to ensure good focus of the particles in the sample plane. A confocal pinhole aperture is shown in this example, which pinhole serves to spatially filter fluorescence from positions above and below the sample plane which might otherwise interfere with measurement of particle fluorescence.

It will be appreciated that the system could be modified in many ways, such as by including additional lasers or fluorescence detectors.

It is understood that multiple methods exist to segment particles from the set of 2-dimensional images described in this disclosure and to calculate fluorescent values and the other measured parameters described in this disclosure. The computer program CellProfiler (https://CellProfiler.org) is one example of software that provides all the tools necessary to perform the segmentation of particles and calculation of fluorescence values and other measured parameters of the particles described herein.

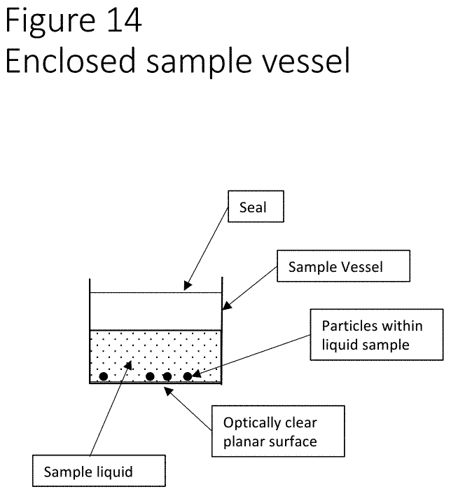

FIG. 14 illustrates an enclosed sample vessel. A seal prevents escape of the sample liquid, thereby preventing contamination of one sample with liquid from other samples or contaminants typically present in a laboratory or clinical setting. This seal also protects laboratory personnel from biohazardous material that could become aerosol or could spill out of the sample vessel. The seal could include a septum (not shown) which admits reagents into the vessel but blocks escape of liquid from the vessel. Alternately, the seal could be installed after all reagents and the sample have been added to the vessel but prior to analysis of the assay. The bottom surface of the vessel is planar and optically clear, enabling analysis of the sample pursuant to the disclosure.

The sealing surface and the vessel walls could be clear or opaque. If the sealing surface is not substantially clear, then only fluorescence and reflected light from particles in the sample could be measured but light transmitted through the sample would not be measurable. It will be appreciated that either configuration allows implementation of the disclosed invention.

This specification is to be construed as illustrative only and is for the purpose of teaching those skilled in the art the manner of carrying out the disclosure. It is to be understood that the forms of the disclosure herein shown and described are to be taken as the presently preferred embodiments. As already stated, various changes may be made in the shape, size and arrangement of components or adjustments made in the steps of the method without departing from the scope of this disclosure. For example, equivalent elements may be substituted for those illustrated and described herein and certain features of the disclosure maybe utilized independently of the use of other features, all as would be apparent to one skilled in the art after having the benefit of this description of the disclosure.

While specific embodiments have been illustrated and described, numerous modifications are possible without departing from the spirit of the disclosure, and the scope of protection is only limited by the scope of the accompanying claims.

1. A method for detection and identification of at least two different kinds of antibodies within a sample comprising:
a. reacting within a sealable reaction vessel having at least one planar, optically clear surface, a liquid biological sample containing at least a first and second type of antibodies wherein each type of antibody is specific to a different capture antigen, and a microsphere solution containing at least a first and second set of microspheres wherein each set of microspheres has been coated with a unique capture antigen;
b. adding at least a first type of fluorescently labeled detection antibodies to the biological sample that binds to at least one isotype or subtype of the first and second type of antibodies present in the sample;
c. sealing the reaction vessel;
d. allowing the sample to incubate with the microsphere solution and detection antibodies for a predetermined period of time;
e. using a laser-scanning cytometer to measure within the reaction vessel the optical properties of the microspheres;
f. distinguishing the first set of microspheres from the second set of microspheres using at least one optical property; and
g. comparing the measured optical properties for each set of microspheres with prior data to infer the concentration level of at least an isotype or subtype of the type of antibody which binds to the capture antigen coated onto that set of microspheres.

2. The method of claim 1 wherein a capture antigen is a protein, peptide, or polysaccharide.

3. The method of claim 1 wherein more than one type of fluorescently labeled detection antibody is added to the sample, each of which detection antibody binds to a different isotype or subtype of antibody and each fluorescently labeled detection antibody is distinguished from other fluorescently labeled detection antibodies by a difference in the intensity of fluorescence emissions at one or more emission wavelengths.

4. The method of claim 3 wherein the measured optical properties for each set of microspheres are compared with prior data to infer concentration level of an isotype or subtype of the first type of antibody and second type of antibody in the sample.

5. The method of claim 1 wherein at least one optical property of the first set of microspheres is distinct from the optical properties of the second set of microspheres by absorption of certain wavelengths of light or fluorescence at a certain wavelength or range of wavelengths.

6. A method for detection and identification of one or more types of antibodies and one or more biomarkers that are not antibodies within a sample comprising:
a. reacting within a reaction vessel having at least one planar, optically clear surface a liquid biological sample containing
at least one type of antibody and at least one biomarker that is not an antibody with
a microsphere solution containing at least one set of microspheres coated with a capture antigen that binds with at least one type of antibodies or at least a separate set of microspheres coated with a capture antigen that binds to a biomarker that is not an antibody;
b. adding at least one type of fluorescently labeled detection antibodies to the biological sample wherein the fluorescently labeled detection antibodies bind with at least one isotype or subtype of the of antibodies present in the sample;
c. adding a fluorescently labeled detection antibody to the biological sample that binds with the biomarker present in the sample that is not an antibody;
d. sealing the reaction vessel;
e. allowing the sample to incubate with the microsphere solution and detection antibodies for a predetermined period of time;
f. using a laser-scanning cytometer to measure optical properties of the microspheres within the reaction vessel;
g. distinguishing the sets of microspheres added to the sample by at least one optical property; and
h. comparing the measured optical properties for each set of microspheres with prior data to infer the concentration level of an isotype or subtype an antibody in the sample or the concentration level in the sample of a biomarker that is not an antibody.

7. The method of claim 6 wherein the capture antigen is a protein, peptide, or polysaccharide.

8. The method of claim 6 wherein more than one type of fluorescently labeled detection antibodies are added to the sample, each type of detection antibodies binds to a different isotype or subtype of antibody and is distinguished from other fluorescently labeled detection antibodies by differing intensity of fluorescence emissions at one or more emission wavelengths.

9. The method of claim 8 wherein the measured optical properties for each set of microspheres are compared with prior data to infer at least one concentration level.

10. The method of claim 6 wherein at least one optical property used to distinguish the sets of microspheres is absorption of light at a certain wavelength or fluorescence at a certain wavelength or range of wavelengths.

11. The method of claim 6 further comprising adding a plurality of sets of microspheres, wherein each set of microspheres is coated with a unique capture antigen that binds with a unique type of antibodies.

12. The method of claim 11 wherein the measured optical properties for each set of microspheres are compared with prior data to infer a presence or concentration level in the sample of at least one isotype or subtype of antibody that binds to the capture antigen coated onto that set of microspheres.

13. The method of claim 6 further comprising adding a plurality of sets of microspheres wherein each set is coated with a unique capture antigen that binds to a biomarker that is not an antibody.

14. The method of claim 13 further comprising measuring the measured optical properties for each set of microspheres and comparing the measured properties with prior data to infer a concentration level in the sample of the biomarker that is not an antibody that binds to the capture antigen coated onto that set of microspheres.
